(19) United States
(12) Patent Application Publication
(10) Pub. No.: US 2024/0398318 A1
(43) Pub. Date: Dec. 5, 2024

(54) Methods, systems, non-transitory computer readable medium, and machines for maintaining augmented telepathic data

(71) Applicant: William Henry Starrett, Jr., Richardson, TX (US)

(72) Inventor: William Henry Starrett, Jr., Richardson, TX (US)

(73) Assignee: William Henry Starrett, Jr., Richardson, TX (US)

(21) Appl. No.: 18/329,482

(22) Filed: Jun. 5, 2023

Related U.S. Application Data
(63) Continuation of application No. 15/299,124, filed on Oct. 20, 2016.
(60) Provisional application No. 62/254,069, filed on Nov. 11, 2015.

(51) Int. Cl. A61B 5/372; A61B 5/00; G16H 20/70

(52) U.S. Cl. CPC A61B 5/372; A61B 5/0006; A61B 5/0022; A61B 5/7267; G16H 20/70; G06Q 20/321; G06Q 50/26

(57) ABSTRACT

Effective augmented telepathic communication is provided as a gadget-free extension of human senses. The conveyance of mental information and cognitive processes to perceive or communicate being made possible by data structures for generating and maintaining representations of biological systems activity with auditory, visual, kinesthetic, tactile, emotion, movement, smell, taste, and concept data in a computing environment. One or more methods include steps for representing brain, nervous system, and sensory systems activity with mental information and cognitive processes as data for incorporating values with applications, systems, or instance relevant computing environments. In preferable embodiments of the present invention, methods also include the association of data representing the objects, elements, assets, acts, conditions, processes, or products of perceiving, cognizing, communicating, experiencing, imagining, remembering, recognizing, judging, reasoning, problem solving, conceptualizing, or planning with mixes of machine learning tasks for human-computer interfacing and communicating using optional devices or acting as a biological computing environment.

### CROSS-REFERENCE TO RELATED APPLICATIONS

This application is a continuation of

Nonprovisional application Ser. No. 15/299,124 filed on 2016 Oct. 20

which claimed the benefit of Provisional Application No. 62/254,069 filed 2015 Nov. 11 and is a divisional application of application Ser. No. 14/921,682, filed 2015 Oct. 23
which claims the benefit of or is a continuation of Provisional Application No. 62/068,463 filed 2014 Oct. 24.

### STATEMENT REGARDING FEDERALLY SPONSORED RESEARCH OR DEVELOPMENT

### Not Applicable

### INVENTOR

William H. Starrett, Jr.

101 S. Coit Ste. 36-222; Richardson, Texas 75080; United States of America 972.783.6947

bill@wideopenmedia.com

### REFERENCE TO SEQUENCE LISTING, A TABLE, OR A COMPUTER PROGRAM LISTING COMPACT DISC APPENDIX

Not Applicable

### STATEMENT REGARDING PRIOR DISCLOSURES BY THE INVENTOR OR A JOINT INVENTOR

This application is a continuation of Nonprovisional application Ser. No. 15/299,124 filed on 2016 Oct. 20. Prior disclosures include Provisional Application No. 62/254,069 filed 2015 Nov. 11; and, application Ser. No. 14/921,682, filed 2015 Oct. 23 which claims the benefit or is a continuation of Provisional Application No. 62/068,463 filed 2014 Oct. 24.

### BACKGROUND OF THE INVENTION

### Technical Field

The present invention relates to the field of computing. More particularly, the present invention relates to methods, systems, non-transitory computer readable medium, and machines for generating, analyzing, extending, communicating, integrating, storing, converting, editing, encoding, or maintaining representations of auditory, visual, kinesthetic, tactile, emotion, concept, movement, smell, taste, and communications data in a computing environment.

### Background Art

Using a machine with a memory, system with a memory, or non-transitory computer readable medium, current techniques related to representing mental information or cognitive processes for perceiving or communicating typically generate or maintain captures of only the broadest view output from voicing, writing, or gesturing interactions as a shortcoming.

Using traditional means, data relating thought, imagery, symbol, or sound is limited to measurement from generalized conscious interactions with gadgetry or accessories that often require batteries or a nearby electric power source.

Data strategies for representations interfacing the human body and mind with computing environments typically require wearable or surgically implanted devices using invasive sensors, probes, or electrodes impacting health or comfort while restricting mobility.

With models containing available data collected external to a body, aggregated representations are limited to rudimentary specialized measurement mixes as broad generalizations at low resolutions. Without acutely representing active and passive user data beyond manual entry or generalized sensor readings lacking explicitness, the limiting of beneficial applications persists.

### BRIEF SUMMARY OF THE INVENTION

Objects of the present invention provide novel methods, systems, non-transitory computer readable medium, and machines for maintaining data relating to effective augmented telepathic communication as a gadget-free extension of human senses.

Objects of the present invention are methods, systems, non-transitory computer readable medium, and machines for generating, analyzing, extending, communicating, integrating, storing, converting, editing, encoding, or maintaining representations of biological systems activity, mental information, and cognitive processes optionally being represented in association with expressions referencing auditory, visual, kinesthetic, tactile, emotion, concept, movement, smell, taste, and communications data in a computing environment.

Aspects of the present invention include novel systems and methods for generating and maintaining data structure representations of mental information or cognitive processes being used for one or more of the following: extending human communication and intelligence; directly perceiving or communicating without optional voicing, writing, or gesturing; augmenting with computing environments; and leveraging external data for answering questions or anticipating needs.

An advantage from generating and maintaining data structures representing active and passive mental information or cognitive processes from preferable embodiments of the present invention is communication with or without subvocalization and without optional voicing, writing, or gesturing.

Advantages of generating and maintaining data structures representing direct sensory perception multiply from the opportunity to interface the human mind and body with computing environments without cables, optional nearby gadgetry, or invasive, uncomfortable, or cumbersome appliances.

In preferable embodiments of the present invention, a computer readable medium (example, for instance, random access memory or a computer disk), a machine with a memory, or a system with a memory, comprises data, code, or both for carrying out methods and maintaining representations of obtained measures as described herein.

### BRIEF DESCRIPTION OF DRAWINGS

A full disclosure including best mode and other features, aspects, and advantages of the present invention, as directed to one of ordinary skill in the art as set forth in the specification, makes reference to the accompanying drawings, wherein:

FIG. 1 is a pictorial diagram illustrating the relative positions of a human eye, optic nerve, brain, and spinal cord as biological system candidates for the granular recording and modeling of activity as spatiotemporal values relating the visually perceived, remembered, or imagined to portable meanings in a preferable embodiment of the present invention;

FIG. 2 is a flowchart diagram illustrating an example for how representations of perception, cognition, and communication, in this example characterized as components of processing visual data, relate in biological systems being represented using a preferable embodiment of the present invention;

FIG. 3 is a flowchart diagram illustrating an example of incorporating mixes of supervised, semi-supervised, unsupervised, or reinforcement learning algorithms for associating representations in one or more embodiment of the present invention;

FIG. 4 is a flowchart diagram illustrating an example of how communication and information data travels when incorporating satellite-based technologies or ambient fields networked, in this example using wireless, Optical Carrier, or satellite-based systems, in a preferable embodiment of the present invention;

FIG. 5 is a system diagram illustrating a representation model of environments for exemplary generating, maintaining, and consuming according to preferable embodiments;

FIG. 6 is a data structure diagram depicting one approach for representing biological computing environment data in a preferable embodiment of the present invention;

FIG. 7 is a pictorial diagram depicting a machine, in this example embodiment, a general purpose computer system capable of supporting a high resolution graphics display device and a cursor pointing device, such as a mouse, on which one preferable embodiment of the present invention may be implemented;

FIG. 8 is a diagram illustrating a non-transitory computer readable medium containing data representing either of, or both of, data structures and program instructions in a preferable embodiment; and

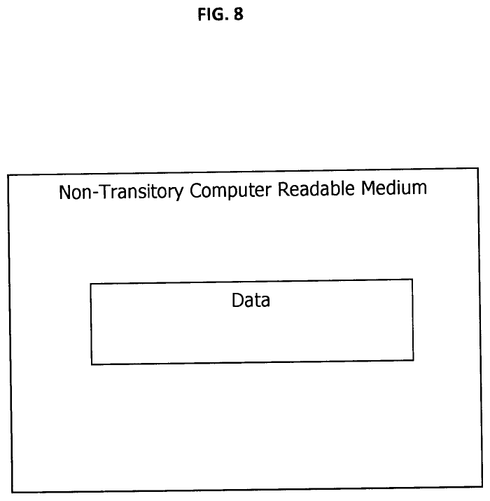

FIG. 9 is a pictorial diagram depicting one exemplary tracking and data relay satellite as implemented in preferable embodiments.

### DETAILED DESCRIPTION OF THE INVENTION

In the nervous system of a biological body, information travels from one cell to another. From the first cell to the next along a given pathway, electrical and chemical signals transport patterns of data.

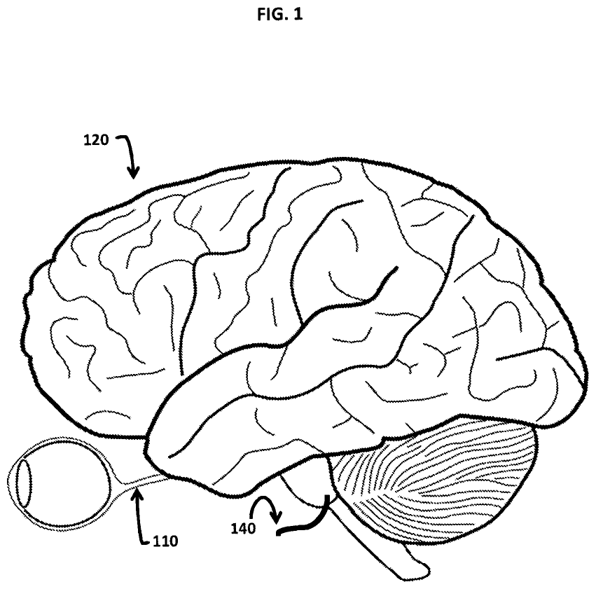

According to contemporary research, a human brain (shown by symbol 120 in FIG. 1) leverages approximately 100 billion nerve cells called neurons. Supported by neuroglia, these neurons can each form thousands of connections for sending or receiving input between them. Combined, each neuron with as many as ten thousand other neurons (or more) transacting through synapse connections, a human brain's networked synaptic count can total well into the trillions.

While synapse gaps between cells measure approximately 0.02 micrometers (20 nanometers), as messages move from one neuron to another, a neuron can fire signals at rates between five and fifty times per second or more. As historically measured from throughout a brain mass, the activity being referred to as neural oscillation, brain waves, or rhythms.

While the brain processes data with assistance from neuron cell transmits, the eyes and ears communicate incoming sensory data to the brain with specialized systems.

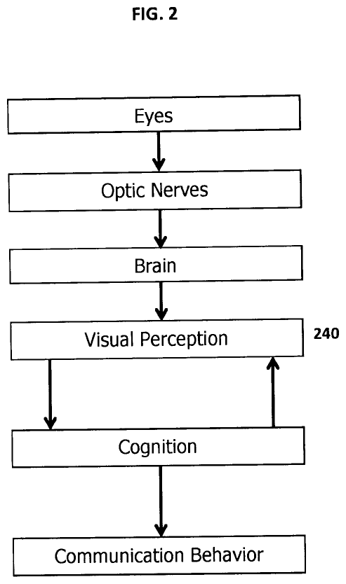

For vision data from the eyes, an optic nerve (noted by symbol 110 in FIG. 1) conveys to the brain from the retina with as many as an approximate 1.5 million nerve fibers. Visual imagery data including the perception of brightness, color, and contrast is signaled to the brain by electrical impulses for processing (represented by symbol 240 in FIG. 2).

For sound data from the ears, the vestibulocochlear (auditory vestibular) nerve connects to the brain (shown by symbol 140 in FIG. 1) for relaying information accumulated by outer, middle, and inner ear physical structures. In addition to sound data carried by the cochlear nerve from the ears, the vestibular nerve helps to extend the senses by relaying inputs relevant to position and balance. Using the vestibular nerve, rotational, linear, and vertical force acceleration data are also made available to the brain.

Much like from the eyes and ears, cranial nerves communicate patterns of data related to eye movement, facial expression, smell, oral sensations like taste, salivation and swallowing, movement in the tongue, shoulder, neck, and head. Each cell connection and nerve fiber communicating decipherable patterns of electrical or chemical information related to events and statuses making it or other surrounding physiological clues a candidate for granular recording and modeling of localized and grouped activity.

Laser scanning and microwave imaging related technologies, with precise remote measurement of distance and time, are employable for modeling ranging data, molecular changes, and chemical reactions. Focused and directed, wavelengths across the electromagnetic spectrum are used for analysis and broadcast to depths through otherwise fragile or impenetrable mass.

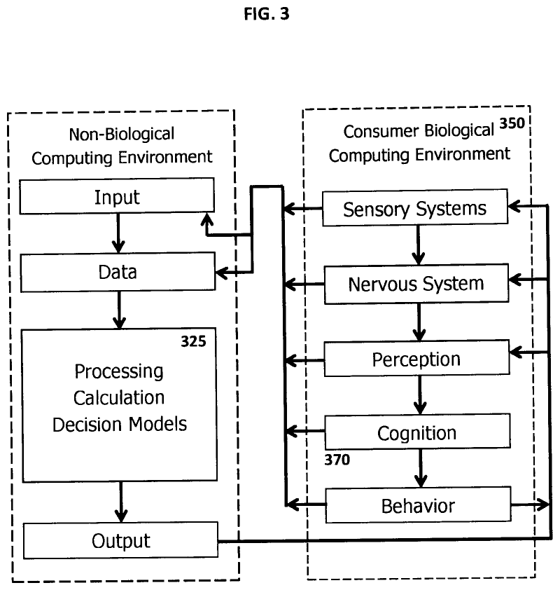

While one embodiment of the present invention could approach remote gathering of the physiological clues of perception and cognitive activity (represented by symbol 370 in FIG. 3) with microscopy techniques, other embodiments may obtain or map required instances at scale down to the nanoscopic through interferometry methods, remote sensing, or similar. Still, particular embodiments of the present invention benefit from other techniques and wavelengths to actively observe and instantly model electrical impulse events, molecular signaling, chemical changes, magnetic properties, or other types of trace occurrences.

In a preferable embodiment of the present invention, when observing momentary brain or nerve fiber activity to measurements at the micrometer, nanometer, or below, the more granularly detailed the measuring and recording of activity in real time, the more apparent the patterns of signaling and trace occurrences through nerve cells and the brain relevant or correlated to the universe of specific actions or instances in an experience (represented by symbol 350 in FIG. 3).

A preferable embodiment of the present invention regards brain or nerve activity and trace occurrences as spatiotemporal event data that allows for analysis with or without mixes of supervised, semi-supervised, unsupervised, and reinforcement machine learning tasks (noted by symbol 325 in FIG. 3). Suites of independent cases associating brain or nerve activity with references to visual, auditory, or other sensory information either being perceived or remembered offer opportunities for the recognition of state or activity patterns matching correlated mental information or cognitive processes for perceiving and communicating.

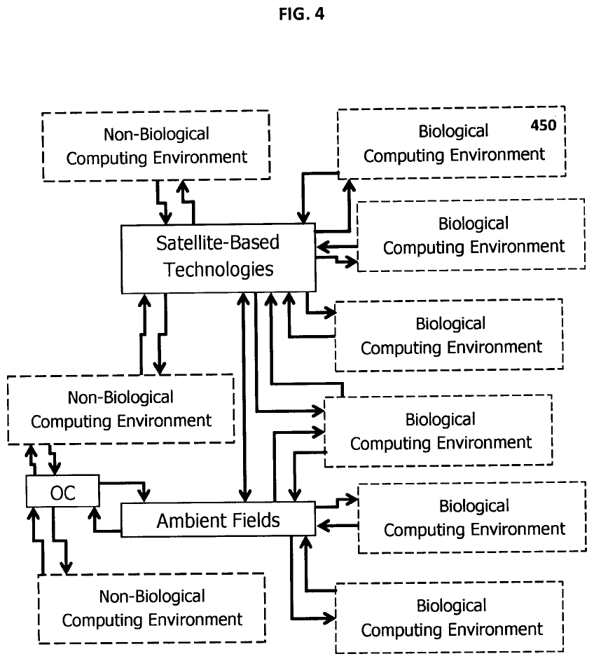

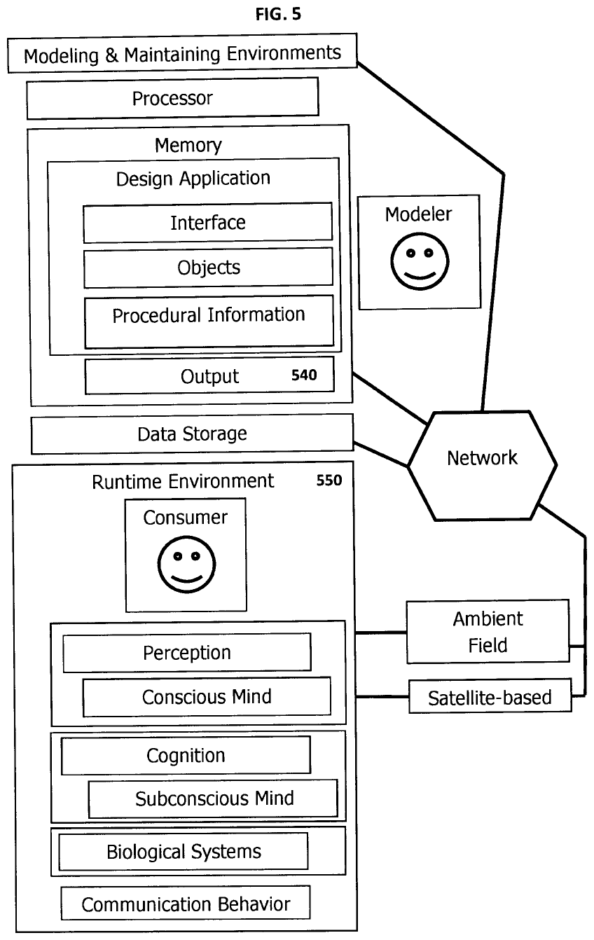

As data, the remote dimensional modeling of real-time brain activity and nerve signaling patterns matched with relevant counterparts as portable extensible meanings, embodiments of the present invention interface in arrangement with biological and non-biological computing environments (noted by symbol 450 in FIG. 4) for effective augmentation and telepathic communication as an extension of human senses (represented by symbol 550 in FIG. 5).

Communication, the extending and amplifying of intelligence, the ability to multiply memory, and instant knowledge is supported by applications (noted by symbol 540 in FIG. 5) offering medical or psychological treatment, augmented reality, access control, situational awareness, decision support, navigation, measurement, calculation, instant language translation tools, self improvement, and personal development strategy management across modalities. Application categories classifying under intended use in preferable embodiments of the present invention include education, entertainment, business, productivity, and science.

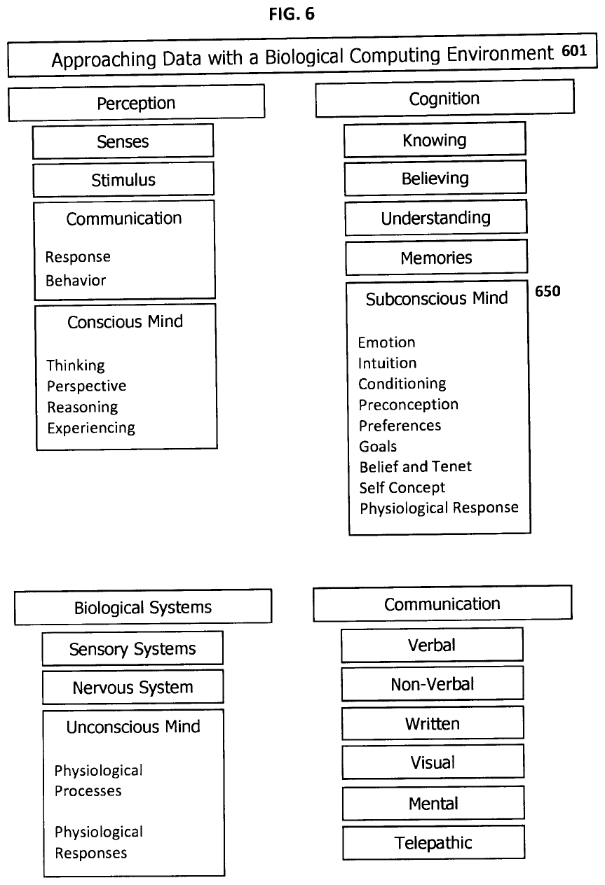

While preferable embodiments of the present invention relates communications patterns between cells in the brain, across nerve fibers, or measurement of trace occurrences from activity with correlating perception data (noted by symbol 601 in FIG. 6) for generating, analyzing, extending, communicating, integrating, storing, converting, editing, encoding, or maintaining representations, returned output to or incoming data to a receiving biological body can chiefly be broadcasted to the activity of the nerves already transporting sensory data instead of the neurons or networks of synapse connections related to processing it.

In preferable embodiments of the present invention, incoming data for sound is patterned for stimulating the ear structures or auditory nerves while, with visual data, imagery being perceived in the imagination, input signals can be patterned to stimulate the optic nerves or eyes. Across modalities, in preferable embodiments of the present invention, the direct subjective conscious interaction with information is optionally being supported or enhanced with the subliminal (noted by symbol 650 in FIG. 6).

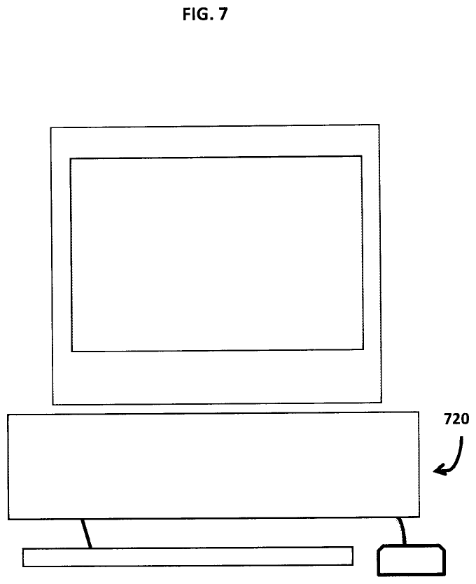

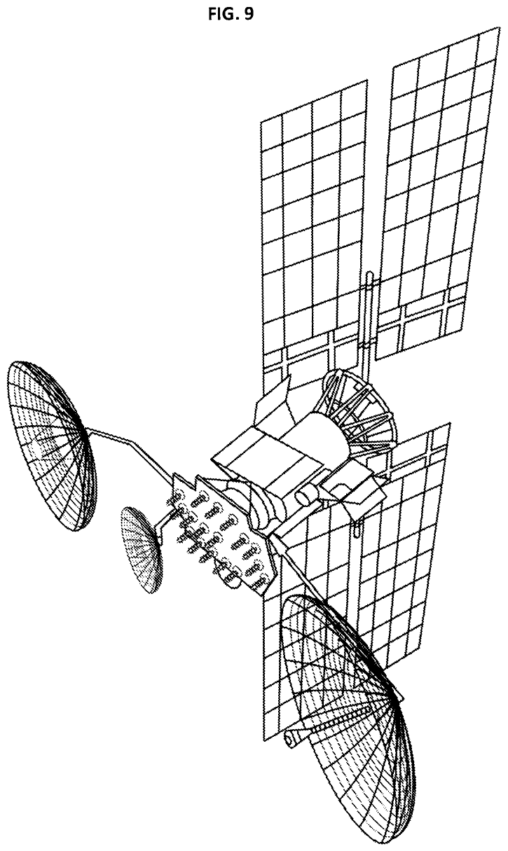

The remote generating and maintaining of data structure representations interfacing a mobile biological body with computing environments (represented by symbol 720 in FIG. 7), at rest or in motion as ideal in most preferable embodiments of the present invention, offered analogously from satellite-based (represented in FIG. 9) technologies for sensing, classifying, and tracking objects as implemented by defense programs. Objectives in preferable embodiments of the present invention, in most cases, are to be well maintained using radar, radio, optics, directed energy, or networks of ambient fields (noted by FIG. 4).

The greatest potentials of present invention preferable embodiments being realized are from the reasoning with ideas, abbreviated time for problem solving, adjusted knowing in preconception and belief based on needs and data, and a refinement of one's behavior and experience toward their personal ideal. The most preferable of all embodiments being ethically maintained only when securely generated or maintained data is received from or broadcasted to individuals with their knowledge and consent under agreement with respect to their privacy (noted by FIG. 5).

1. A non-transitory computer readable medium containing data representing either of or both data structures and program instructions for generating, analyzing, extending, communicating, integrating, storing, converting, editing, encoding, or maintaining said data structures representing
one or more unit of category Nervous System depicting referring expressions relating to nervous system cells, nerves, tissue, electrical or chemical impulses, and trace occurrences related to signaling the communication of information and its processing in a biological body optionally with
zero, one, or more unit of category Sensory System depicting referring expressions relating to sensory systems cells, nerves, tissue, electrical or chemical impulses, and trace occurrences related to signaling the communication of sensory information for its interpretation or processing in a biological body and
zero, one, or more unit of category Brain and Nerve Activity optionally depicting referring expressions associating Nervous System category units with Sensory System category units wherein
zero, one, or more Brain and Nerve Activity,
zero, one, or more Sensory System, and
one or more Nervous System units with Sensory System and Nervous System units optionally relating as Brain and Nerve Activity units being associated with one or more unit in at least one of the following categories:
Communication depicting referring expressions relating to the conveyance of ideas as sound, visual imagery, text, concept, or feelings;
Cognition depicting referring expressions relating to considering, knowing, understanding, or believing;
Perception depicting referring expressions relating to sensing, perceiving, observing, or becoming aware;
Experience depicting referring expressions relating to feeling or reflecting on abstractions;
Imagery depicting referring expressions relating to the visually perceived, remembered, or imagined;
Sound depicting referring expressions relating to the auditorily perceived, remembered, or imagined;
Symbol depicting referring expressions relating to meanings;
Stimulus depicting referring expressions relating to evoking actions and conditions;
Behavior depicting referring expressions relating to acting as or reacting to a stimulus; and
People depicting referring expressions relating to community groups and individuals
wherein each Brain and Nerve Activity, Sensory System, Nervous System, Communication, Cognition, Perception, Experience, Imagery, Sound, Symbol, Stimulus, Behavior, and People category unit consisting of
zero, one, or more members with each member describing one or more object, element, asset, act, condition, process, or product representing zero, one, or more event, status, location, or hierarchical coordinate system and having zero, one, or more relationship, reference, property, description, or dimension of interest wherein
data structures representing one or more unit in one or more category being generated using
one or more referring expression and zero, one, or more hierarchical coordinate system by an optional method comprising the steps of:
analyzing one or more body;
obtaining information about one or more body; and
generating one or more representation.

2. The non-transitory computer readable medium of claim 1, wherein
the analyzing step of the optional method comprising
the examination of any object, element, asset, act, condition, process, or product of the biological systems, communication, or behavior of
one or more body using zero, one, or a plurality of wearable or surgically implanted device, sensor, probe, or electrode with
zero, one, or more proximity to one or more body using
zero, one, or more wearable or surgically implanted device, sensor, probe, or electrode.

3. The non-transitory computer readable medium of claim 2, wherein
data structures being generated or maintained comprising at least one of the following:
data representing information obtained from
one or more body with zero, one, or more representation of a vantage optionally being associated with referring expressions relating the objects, elements, assets, acts, conditions, processes, or products of perceiving, cognizing, communicating, experiencing, imagining, remembering, recognizing, thinking, judging, reasoning, problem solving, conceptualizing, or planning with zero, one, or more representation of a vantage;
data representing one or more referring expression relating one or more representation of:
(visual, auditory, or kinesthetic data obtained from one or more body with zero, one, or more representation of a vantage;
non-verbal, verbal, perceived, remembered, or imagined communication with zero, one, or more representation of a vantage;
measurement, activity, or condition associated with
zero, one, or more location of one or more of the following obtained from one or more perceiving, cognizing, experiencing, remembering, or imagining body:
biological systems, sensory systems, nervous system, brain cells, nerves, tissue, electrical impulse events, molecular signaling, chemical changes, magnetic properties, and trace occurrences;
or
text, sound, visual imagery, thought, idea, meaning, relationship, feeling, and communication obtained from one or more perceiving, cognizing, experiencing, remembering, or imagining body with zero, one, or more representation of a vantage);
data representing one or more parameter in stimulation transmission signal formatting instructions, wherein
stimulation transmission signal comprising one or more referring expression relating:
(one or more representation of text, sound, visual imagery, thought, idea, meaning, relationship, feeling, vantage, or communication obtained from one or more perceiving, cognizing, experiencing, remembering, or imagining body;
perception or cognition directly to biological systems of one or more body;
visual, auditory, or kinesthetic perception or cognition directly to biological systems of one or more body;
non-verbal, verbal, perceived, remembered, or imagined communication directly to biological systems of one or more body;
the objects, elements, assets, acts, conditions, processes, or products of perceiving, experiencing, remembering, imagining, or cognizing using sound, visual imagery, text, and feelings directly to biological systems of one or more body;
the objects, elements, assets, acts, conditions, processes, or products of perceiving, cognizing, communicating, experiencing, imagining, remembering, recognizing, thinking, judging, reasoning, problem solving, conceptualizing, or planning with zero, one, or more representations of a vantage;
or
subliminal information optionally being combined with auditory, visual, kinesthetic, tactile, emotion, concept, movement, smell, taste, or communications data directly to biological systems of one or more consuming or interacting body);
data representing one or more referring expression relating:
(active and passive user information;
one or more representation comprising information obtained about one or more body using satellite-based technologies, ambient fields, microscopy techniques, interferometry methods, remote sensing, radar, radio, optics, directed energy, or tracking technologies;
one or more interacting or interfacing body with the objects, elements, assets, acts, conditions, processes, or products of one or more computing environment using zero, one, or more hardware component or device item;
communication without optional subvocalization, voicing, writing, or gesturing;
one or more body with data structures or program instructions performing one or more financial transaction using one or more program, application, interface, or marketplace;
one or more body with data structures or program instructions performing one or more of the following tasks:
analyzing, extending, communicating, integrating, storing, converting, editing, or encoding;
one or more body with data structures or program instructions interfacing one or more human user participating as one or more biological computing environment in networking arrangement with zero, one, or more computing environment;
one or more body with data structures or program instructions interfacing one or more user interacting with one or more computing environment using zero localized input devices, zero, one, or more thought, zero, one, or more voiced command, and zero, one, or more gesture;
one or more representation of one or more object, element, asset, act, condition, process, or product of data being associated by zero, one, or more machine learning task classified under zero, one, or more of the following category types:
supervised, semi-supervised, unsupervised, and reinforcement;
or
representations of communication, sensory or nervous system information, perception, cognition, experiences, stimulus, behavior, or relevant conditions with one or more referring expression from first-person, second-person, or third-person perspectives in the manner of zero, one, or more of each of the following:
graphically, programmatically, computationally, textually, numerically, symbolically, audibly, sequentially, or conceptually);
and
data representing one or more referring expression relating at least one body with data structures or program instructions interfacing at least one body with one or more computing environment returning output from, associating one or more representation with output from, or executing at least one of the following process activity types:
(performing tasks supporting communication, search, education, entertainment, research, navigation, measurement, calculation, business, productivity, language tools and translation, song recognition, augmented reality, situational awareness, decision support, medical or psychological treatment, social information interpretation, law enforcement, military, security, surveillance, authorization, access control, building automation, financial transaction, instant knowledge, memory extension, intelligence amplification, graphic design, three dimensional modeling, three dimensional printing, dreams, public speaking, performance, self improvement, or personal development;
biometric measurement of the face, ear, eye, iris, retina, fingerprint, finger or hand geometry, gait, odor, or voice;
locating, identifying, verifying, classifying, profiling, or recognizing of objects, structures, groups, or individuals with zero, one, or more of the following:
facial expression, body language, posture, gesture, tone, proximity, signature or handwriting, typing, writing style, word or phrase choice, registration information, or license plate;
or
computer vision, pattern recognition, shape recognition, object recognition, biometric measurement, speech or voice recognition, geography, proximity, character recognition, or eye tracking).

4. The non-transitory computer readable medium of claim 3, wherein
data structures representing
one or more unit in at least one category being associated by either the means of
one or more machine learning task classified under zero, one, or more of the following category types:
supervised, semi-supervised, unsupervised, and reinforcement
or
an optional method comprising the steps of:
analyzing
at least one category unit member using one or more referring expression and the optional means of
zero, one, or more machine learning task classified under zero, one, or more of the following category types:
supervised, semi-supervised, unsupervised, and reinforcement;
obtaining zero, one, or more referring expression; and
generating one or more representation.

5. A machine having a memory containing data representing either of or both data structures and program instructions for generating, analyzing, extending, communicating, integrating, storing, converting, editing, encoding, or maintaining said data structures representing one or more unit in at least one of the following categories:
Mental Information depicting referring expressions relating to the known, believed, and understood;
Cognitive Processes depicting referring expressions relating to sensing, observing, considering, imagining, feeling, experiencing, remembering, and committing to memory;
Communications Information depicting referring expressions relating to conveying ideas, acting as a stimulus, and responding to stimulus;
Biological Systems depicting referring expressions relating to application relevant organs, divisions, tissue, cells, nerves, processes, or activities in a biological body;
Biological Systems Activity depicting referring expressions relating to the application relevant processes or activity pertaining to organs, divisions, tissue, cells, nerves, electrical or chemical impulses, and trace occurrences related to signaling the communication of information and its processing in a biological body;
People depicting referring expressions relating to community groups and individuals; and
Stimulus depicting referring expressions relating to evoking actions and conditions;
wherein each Mental Information, Cognitive Processes, Communications Information, Biological Systems, Biological Systems Activity, People, and Stimulus category unit consisting of
zero, one, or more members with each member describing one or more object, element, asset, act, condition, process, or product and representing
zero, one, or more event, status, location, or hierarchical coordinate system and having
zero, one, or more relationship, reference, property, description, or dimension of interest.

6. The machine of claim 5, wherein
data structures representing at least one unit in one or more category being generated using
one or more referring expression and zero, one, or more hierarchical coordinate system by an optional method comprising the steps of:
analyzing one or more body;
obtaining information about one or more body; and
generating one or more representation.

7. The machine of claim 6, wherein
data structures being generated or maintained comprising at least one of the following:
data representing information obtained from
one or more body with
zero, one, or more representation of a vantage
optionally being associated with referring expressions relating the objects, elements, assets, acts, conditions, processes, or products of perceiving, cognizing, communicating, experiencing, imagining, remembering, recognizing, thinking, judging, reasoning, problem solving, conceptualizing, or planning with
zero, one, or more representation of a vantage;
data representing one or more referring expression relating one or more representation of:
(visual, auditory, or kinesthetic data obtained from one or more body with zero, one, or more representation of a vantage;
non-verbal, verbal, perceived, remembered, or imagined communication with zero, one, or more representation of a vantage;
measurement, activity, or condition associated with zero, one, or more location of one or more of the following obtained from one or more perceiving, cognizing, experiencing, remembering, or imagining body:
biological systems, sensory systems, nervous system, brain cells, nerves, tissue, electrical impulse events, molecular signaling, chemical changes, magnetic properties, and trace occurrences;
or
text, sound, visual imagery, thought, idea, meaning, relationship, feeling, and communication obtained from
one or more perceiving, cognizing, experiencing, remembering, or imagining body with zero, one, or more representation of a vantage);
data representing one or more parameter in stimulation transmission signal formatting instructions, wherein
stimulation transmission signal comprising one or more referring expression relating:
(one or more representation of text, sound, visual imagery, thought, idea, meaning, relationship, feeling, vantage, or communication obtained from one or more perceiving, cognizing, experiencing, remembering, or imagining body;
perception or cognition directly to biological systems of one or more body;
visual, auditory, or kinesthetic perception or cognition directly to biological systems of one or more body;
non-verbal, verbal, perceived, remembered, or imagined communication directly to biological systems of one or more body;
the objects, elements, assets, acts, conditions, processes, or products of perceiving, experiencing, remembering, imagining, or cognizing using sound, visual imagery, text, and feelings directly to biological systems of one or more body;
the objects, elements, assets, acts, conditions, processes, or products of perceiving, cognizing, communicating, experiencing, imagining, remembering, recognizing, thinking, judging, reasoning, problem solving, conceptualizing, or planning with zero, one, or more representations of a vantage;
or
subliminal information optionally being combined with auditory, visual, kinesthetic, tactile, emotion, concept, movement, smell, taste, or communications data directly to biological systems of one or more consuming or interacting body);
data representing one or more referring expression relating:
(active and passive user information;
one or more representation comprising information obtained about one or more body using satellite-based technologies, ambient fields, microscopy techniques, interferometry methods, remote sensing, radar, radio, optics, directed energy, or tracking technologies;
one or more interacting or interfacing body with the objects, elements, assets, acts, conditions, processes, or products of one or more computing environment using zero, one, or more hardware component or device item;
communication without optional subvocalization, voicing, writing, or gesturing;
one or more body with data structures or program instructions performing one or more financial transaction using one or more program, application, interface, or marketplace;
one or more body with data structures or program instructions performing one or more of the following tasks:
analyzing, extending, communicating, integrating, storing, converting, editing, or encoding;
one or more body with data structures or program instructions interfacing one or more human user participating as one or more biological computing environment in networking arrangement with zero, one, or more computing environment;
one or more body with data structures or program instructions interfacing one or more user interacting with one or more computing environment using zero localized input devices, zero, one, or more thought, zero, one, or more voiced command, and zero, one, or more gesture;
one or more representation of one or more object, element, asset, act, condition, process, or product of data being associated by zero, one, or more machine learning task classified under
zero, one, or more of the following category types:
supervised, semi-supervised, unsupervised, and reinforcement;
or
representations of communication, sensory or nervous system information, perception, cognition, experiences, stimulus, behavior, or relevant conditions with one or more referring expression from first-person, second-person, or third-person perspectives in the manner of zero, one, or more of each of the following:
graphically, programmatically, computationally, textually, numerically, symbolically, audibly, sequentially, or conceptually);
and
data representing one or more referring expression relating at least one body with data structures or program instructions interfacing at least one body with one or more computing environment returning output from, associating one or more representation with output from, or executing at least one of the following process activity types:
(performing tasks supporting communication, search, education, entertainment, research, navigation, measurement, calculation, business, productivity, language tools and translation, song recognition, augmented reality, situational awareness, decision support, medical or psychological treatment, social information interpretation, law enforcement, military, security, surveillance, authorization, access control, building automation, financial transaction, instant knowledge, memory extension, intelligence amplification, graphic design, three dimensional modeling, three dimensional printing, dreams, public speaking, performance, self improvement, or personal development;
biometric measurement of the face, ear, eye, iris, retina, fingerprint, finger or hand geometry, gait, odor, or voice;
locating, identifying, verifying, classifying, profiling, or recognizing of objects, structures, groups, or individuals with zero, one, or more of the following:
facial expression, body language, posture, gesture, tone, proximity, signature or handwriting, typing, writing style, word or phrase choice, registration information, or license plate;
or
computer vision, pattern recognition, shape recognition, object recognition, biometric measurement, speech or voice recognition, geography, proximity, character recognition, or eye tracking).

8. The machine of claim 7, wherein
the analyzing step of the optional method comprising
the examination of any object, element, asset, act, condition, process, or product of biological systems, communication, stimulus, or behavior of
one or more bodies using zero, one, or a plurality of wearable or surgically implanted device, sensor, probe, or electrode with zero, one, or more proximity to
one or more body using zero, one, or more wearable or surgically implanted device, sensor, probe, or electrode.

9. The machine of claim 8, wherein
data structures representing one or more unit in at least one category being associated by either the means of one or more machine learning task classified under zero, one, or more of the following category types:
supervised, semi-supervised, unsupervised, and reinforcement
or
an optional method comprising the steps of:
analyzing
one or more category unit members using one or more referring expression and the optional means of zero, one, or more machine learning task classified under zero, one, or more of the following category types:
supervised, semi-supervised, unsupervised, and reinforcement;
obtaining zero, one, or more referring expression; and
generating one or more representation.

10. A system having a memory containing data representing either of or both data structures and program instructions for generating, analyzing, extending, communicating, integrating, storing, converting, editing, encoding, or maintaining said data structures representing one or more unit in at least one of the following categories:
Brain Activity depicting referring expressions relating brain cells, nerves, tissue, electrical or chemical impulses, and trace occurrences related to signaling the communication of sensory, bodily, mental, and cognitive information and its processing in the brain of a biological body;
Nerve Activity depicting referring expressions relating cells, nerves, tissue, electrical or chemical impulses, and trace occurrences related to signaling the communication of sensory and bodily information and its processing outside of the brain of a biological body;
Physical Structure depicting referring expressions relating to proximity or application relevant organs, divisions, tissue, cells, nerves and the imparting references of each constituent;
Communication depicting referring expressions relating to the conveyance of ideas, acting as a stimulus, or reacting to a stimulus;
Thought depicting referring expressions relating to perceiving, experiencing, considering, and cognizing sensory information, knowledge, abstractions, or stimulus relevant mental actions;
Imagery depicting referring expressions relating to the visually perceived, remembered, or imagined;
Sound depicting referring expressions relating to the auditorily perceived, remembered, or imagined;
Symbol depicting referring expressions relating to perceiving, imagining, experiencing, and cognizing meanings;
People depicting referring expressions relating to community groups and individuals;
and
Stimulus depicting referring expressions relating to evoking actions and conditions;
wherein each Brain Activity, Nerve Activity, Physical Structures, Communication, Thought, Imagery, Sound, Symbol, People, and Stimulus category unit consisting of
zero, one, or more member with each member describing
one or more object, element, asset, act, condition, process, or product and representing zero, one, or more event, status, location, or hierarchical coordinate system
and
having zero, one, or more relationship, reference, property, description, or dimension of interest.

11. The system of claim 10, wherein
data structures representing at least one unit in one or more category being generated using one or more referring expression and zero, one, or more hierarchical coordinate system by an optional method comprising the steps of:
analyzing one or more body;
obtaining information about one or more body; and
generating one or more representation.

12. The system of claim 11, wherein
data structures being generated or maintained comprising at least one of the following:
data representing information obtained from one or more body with zero, one, or more representation of a vantage optionally being associated with referring expressions relating the objects, elements, assets, acts, conditions, processes, or products of perceiving, cognizing, communicating, experiencing, imagining, remembering, recognizing, thinking, judging, reasoning, problem solving, conceptualizing, or planning with zero, one, or more representation of a vantage;
data representing one or more referring expression relating one or more representation of:
(visual, auditory, or kinesthetic data obtained from one or more body with zero, one, or more representation of a vantage;
non-verbal, verbal, perceived, remembered, or imagined communication with zero, one, or more representation of a vantage;
measurement, activity, or condition associated with zero, one, or more location of one or more of the following obtained from one or more perceiving, cognizing, experiencing, remembering, or imagining body:
biological systems, sensory systems, nervous system, brain cells, nerves, tissue, electrical impulse events, molecular signaling, chemical changes, magnetic properties, and trace occurrences;
or
text, sound, visual imagery, thought, idea, meaning, relationship, feeling, and communication obtained from one or more perceiving, cognizing, experiencing, remembering, or imagining body with zero, one, or more representation of a vantage);
data representing one or more parameter in stimulation transmission signal formatting instructions, wherein
stimulation transmission signal comprising one or more referring expression relating:
(one or more representation of text, sound, visual imagery, thought, idea, meaning, relationship, feeling, vantage, or communication obtained from one or more perceiving, cognizing, experiencing, remembering, or imagining body;
perception or cognition directly to biological systems of one or more body;
visual, auditory, or kinesthetic perception or cognition directly to biological systems of one or more body;
non-verbal, verbal, perceived, remembered, or imagined communication directly to biological systems of one or more body;
the objects, elements, assets, acts, conditions, processes, or products of perceiving, experiencing, remembering, imagining, or cognizing using sound, visual imagery, text, and feelings directly to biological systems of one or more body;
the objects, elements, assets, acts, conditions, processes, or products of perceiving, cognizing, communicating, experiencing, imagining, remembering, recognizing, thinking, judging, reasoning, problem solving, conceptualizing, or planning with zero, one, or more representations of a vantage;
or
subliminal information optionally being combined with auditory, visual, kinesthetic, tactile, emotion, concept, movement, smell, taste, or communications data directly to biological systems of one or more consuming or interacting body);
data representing one or more referring expression relating:
(active and passive user information;
one or more representation comprising information obtained about one or more body using satellite-based technologies, ambient fields, microscopy techniques, interferometry methods, remote sensing, radar, radio, optics, directed energy, or tracking technologies;
one or more interacting or interfacing body with the objects, elements, assets, acts, conditions, processes, or products of one or more computing environment using zero, one, or more hardware component or device item;
communication without optional subvocalization, voicing, writing, or gesturing;
one or more body with data structures or program instructions performing one or more financial transaction using one or more program, application, interface, or marketplace;
one or more body with data structures or program instructions performing one or more of the following tasks:
analyzing, extending, communicating, integrating, storing, converting, editing, or encoding;
one or more body with data structures or program instructions interfacing one or more human user participating as one or more biological computing environment in networking arrangement with zero, one, or more computing environment;
one or more body with data structures or program instructions interfacing one or more user interacting with one or more computing environment using zero localized input devices, zero, one, or more thought, zero, one, or more voiced command, and zero, one, or more gesture;
one or more representation of one or more object, element, asset, act, condition, process, or product of data being associated by zero, one, or more machine learning task classified under zero, one, or more of the following category types:
supervised, semi-supervised, unsupervised, and reinforcement;
or
representations of communication, sensory or nervous system information, perception, cognition, experiences, stimulus, behavior, or relevant conditions with one or more referring expression from first-person, second-person, or third-person perspectives in the manner of zero, one, or more of each of the following:
graphically, programmatically, computationally, textually, numerically, symbolically, audibly, sequentially, or conceptually);
and
data representing one or more referring expression relating at least one body with data structures or program instructions interfacing at least one body with one or more computing environment returning output from, associating one or more representation with output from, or executing at least one of the following process activity types:
(performing tasks supporting communication, search, education, entertainment, research, navigation, measurement, calculation, business, productivity, language tools and translation, song recognition, augmented reality, situational awareness, decision support, medical or psychological treatment, social information interpretation, law enforcement, military, security, surveillance, authorization, access control, building automation, financial transaction, instant knowledge, memory extension, intelligence amplification, graphic design, three dimensional modeling, three dimensional printing, dreams, public speaking, performance, self improvement, or personal development;
biometric measurement of the face, ear, eye, iris, retina, fingerprint, finger or hand geometry, gait, odor, or voice;
locating, identifying, verifying, classifying, profiling, or recognizing of objects, structures, groups, or individuals with zero, one, or more of the following:
facial expression, body language, posture, gesture, tone, proximity, signature or handwriting, typing, writing style, word or phrase choice, registration information, or license plate;
or
computer vision, pattern recognition, shape recognition, object recognition, biometric measurement, speech or voice recognition, geography, proximity, character recognition, or eye tracking).

13. The system of claim 12, wherein
the analyzing step of the optional method comprising
the examination of any object, element, asset, act, condition, process, or product of biological systems, communication, stimulus, or behavior of one or more body using
zero, one, or a plurality of wearable or surgically implanted device, sensor, probe, or electrode with
zero, one, or more proximity to one or more body using
zero, one, or more wearable or surgically implanted device, sensor, probe, or electrode.

14. The system of claim 13, wherein
data structures representing one or more unit in at least one category being associated by either
the means of one or more machine learning task classified under zero, one, or more of the following category types:
supervised, semi-supervised, unsupervised, and reinforcement or an optional method comprising the steps of:
analyzing
one or more category unit members using one or more referring expression and the optional means of zero, one, or more machine learning task classified under zero, one, or more of the following category types:
supervised, semi-supervised, unsupervised, and reinforcement;
obtaining zero, one, or more referring expression; and
generating one or more representation.
